(19) United States
(12) Patent Application Publication
(10) Pub. No.: US 2017/0333424 A1
(43) Pub. Date: Nov. 23, 2017

(54) Amorphous Vortioxetine Hydrobromide

(71) Applicant: Hexal AKTIENGESELLSCHAFT, Holzkirchen (DE)

(72) Inventor: Renate Oppelt, Holzkirchen (DE)

(73) Assignee: Hexal AG, Holzkirchen (DE)

(21) Appl. No.: 15/520,868

(22) PCT Filed: Oct. 23, 2015
(86) PCT No.: PCT/EP2015/074607

§ 371 Date: Apr. 21, 2017

(30) Foreign Application Priority Data
Oct. 24, 2014 (EP) 14190357.5

(51) Int. Cl. A61K 31/495; A61K 9/14; A61K 9/20

(52) U.S. Cl. CPC A61K 31/495; A61K 9/2054; A61K 9/143; A61K 9/146; A61K 9/2031

(57) ABSTRACT

The present invention relates to a pharmaceutical composition comprising amorphous vortioxetine hydrobromide, a process for the preparation thereof, use thereof and a method for stabilizing said pharmaceutical composition.

The present invention relates to a pharmaceutical composition comprising amorphous vortioxetine hydrobromide, a process for the preparation thereof, use thereof and a method for stabilizing vortioxetine hydrobromide in a pharmaceutical composition.

Vortioxetine (1-[2-(2,4-dimethyl-phenylsulfanyl)-phenyl]-piperazine) belongs to a chemical class of psychotropics, the bis-aryl-sulfanyl amines, which is structurally different from all currently known psychotropics. Vortioxetine (also known under the name Lu-AA21004 and the tradename BRINTELLIX®), an oral antidepressant 5-HT transporter inhibitor, is indicated in the US for the treatment of major depressive disorder (MDD), In the EU, the drug is indicated for the treatment of adults with major depressive episodes. Development in other psychiatric indications is ongoing.

The marketed vortioxetine immediate-release film-coated tablets contain the hydrobromide salt of the drug in the crystalline beta form. A method of producing this crystalline form is disclosed in WO 2007/144005. The hydrobromide salts of vortioxetine usually are in crystalline form. The solubility of vortioxetine (as free substance) in water is very low (0.1 mg/ml in water). The solubility of vortioxetine hydrobromide salts is slightly enhanced, but still relatively poor. For instance, according to WO 2007/144005 the water-solubility of the beta form is 1.2 mg/ml. This increase in water solubility is significant but still not optimal. A further increase in the water solubility (and thus bioavailability) would thus be advantageous.

It is known that very often the amorphous forms of chemical compounds have a higher solubility than crystalline forms. However, it is difficult to provide salts of vortioxetine and in particular the hydrobromide salt (which is contained in the marketed formulation) in amorphous form and pharmaceutical compositions containing amorphous vortioxetine hydrobromide have the problem that part or all of the vortioxetine hydrobromide tend to crystallize during storage. It is not acceptable that the physical form of an active ingredient changes during the lifetime of a pharmaceutical composition since the physical form of the active ingredient influences the solubility and thus also the bioavailability.

The non-prepublished copending patent application PCT/EP2014/058546 tries to solve this problem by associating amorphous vortioxetine hydrobromide with an adsorbent which is selected from an anorganic material, in particular silica gel.

According to the Biopharmaceutics Classification System (BCS) vortioxetine hydrobromide is classified in class II, which means that the drug substance has high permeability, but low solubility. Accordingly, the bioavailability is limited by its solution rate.

WO 2005/039551 describes solid solutions of HIV protease inhibitors comprising a water-soluble polymer and a surfactant. According to WO 2005/039551, the described dosage forms exhibit a high attainable AUC (area under the curve), high attainable Cₘₐₓ (maximum plasma concentration) and a low Tₘₐₓ (time to reach maximum plasma concentration). However, the water-soluble polymer and the surfactant have to possess specific properties, such as a Tg (glass transition temperature) of at least 50° C. for the water soluble polymer and an HLB of 4-10, preferably 7-9, for the surfactant.

Given the relatively poor solubility of vortioxetine, there is a need for vortioxetine-containing pharmaceutical compositions that make the active substance available in a form that is as soluble as possible. There is also a need for improvement of the dissolution rate and the bioavailability of the active substance. Nevertheless, the active substance must be in a form which is reproducible and stable over the lifetime of a pharmaceutical composition.

It has now been unexpectedly found that vortioxetine in the form of the better soluble vortioxetine hydrobromide can be provided in an even better soluble amorphous form of vortioxetine hydrobromide if the amorphous vortioxetine hydrobromide is provided in the form of a solid solution or solid dispersion. Unexpectedly, the amorphous vortioxetine hydrobromide is highly stable both physically and chemically when it is formulated as a solid solution or solid dispersion which allows the preparation of pharmaceutical compositions in which the amorphous vortioxetine hydrobromide does not change its physical or chemical state during the lifetime of the pharmaceutical composition which is at least one year, preferably at least 18 months, more preferably at least two years.

The present invention therefore provides a pharmaceutical composition comprising a solid solution or solid dispersion of amorphous vortioxetine hydrobromide in at least one organic carrier.

The present invention also provides a process for the preparation of the pharmaceutical composition comprising a solid solution or solid dispersion of amorphous vortioxetine hydrobromide in at least one organic carrier, comprising a process step selected from (i) subjecting vortioxetine hydrobromide, the at least one organic carrier and optional further ingredients to solvent-evaporation; (ii) subjecting vortioxetine hydrobromide, the at least one organic carrier and optional further ingredients to melt extrusion; and (iii) spray drying a solution or dispersion comprising vortioxetine hydrobromide, the at least one organic carrier and optional further ingredients; in order to obtain a solid solution or solid dispersion.

The present invention also provides a pharmaceutical composition comprising a solid solution or solid dispersion of amorphous vortioxetine hydrobromide in at least one organic carrier for use in the treatment of a disease selected from affective disorders, depression, major depressive disorder, postnatal depression, depression associated with bipolar disorder, Alzheimer's disease, psychosis, cancer, age or Parkinson's disease, anxiety, general anxiety disorder, social anxiety disorder, obsessive compulsive disorder, panic disorder, panic attacks, phobia, social phobia, agoraphobia, stress urinary incontinence, emesis, irritable bowel syndrome, eating disorders, chronic pain, partial responders, treatment resistant depression, Alzheimer's disease, cognitive impairment, attention deficit hyperactivity disorder, melancholia, posttraumatic stress disorder, hot flushes, sleep apnea, alcohol, nicotine or carbohydrate craving, substance abuse and alcohol or drug abuse.

The present invention also provides a method for stabilizing amorphous vortioxetine hydrobromide in a pharmaceutical composition, characterized in that the amorphous vortioxetine hydrobromide is formulated in a solid solution or solid dispersion in at least one organic carrier.

The term “solid dispersion” as used herein defines a system in a solid state (as opposed to a liquid or gaseous state) comprising at least two components, wherein one component is dispersed evenly throughout the other component or components. In particular, the active ingredient is dispersed in at least one organic carrier. The term “solid dispersion” encompasses systems having small particles, typically of less than 1 μm in diameter, of one phase dispersed in another phase. When said dispersion of the components is such that the system is chemically and physically uniform or homogenous throughout or consists of one phase (as defined in thermodynamics), such a solid dispersion will be called a “solid solution”. A “solid solution” can also be considered as a system in a solid state wherein the drug is molecularly dispersed within at least one organic carrier.

In the present case the active ingredient vortioxetine hydrobromide in amorphous form constitutes the small particles in a solid dispersion. The amorphous vortioxetine hydrobromide can either be dispersed or dissolved within the at least one organic carrier as such or the amorphous form of vortioxetine hydrobromide can be associated with an anorganic carrier, in particular silicon dioxide and particles of the amorphous vortioxetine hydrobromide adsorbed to the inorganic carrier are dispersed within the at least one organic carrier.

As used herein, the term “amorphous” means a solid body devoid of long-range crystalline order. Such a lack of crystalline order can be detected and monitored, e.g., by X-ray diffraction (XRD), FT-Raman spectroscopy, and differential scanning calorimetry (DSC).

As used herein, the phrase “amorphous vortioxetine hydrobromide” means the vortioxetine hydrobromide contained in the amorphous solid solution or solid dispersion is in the amorphous state, e.g., there is a minimum of 95% of vortioxetine hydrobromide in the amorphous state in the solid solution or solid dispersion, preferably 98% and more preferably 99% or more, or even 100%.

Unless otherwise noted or obvious in the circumstances, percentage terms used herein express weight/weight percentages.

The particle size in the solid dispersion can be measured e.g. with the technique of dynamic light scattering, e.g. with Zetasizer Nano S, Malvern Instruments Ltd., Worcestershire, UK or a similar instrument of the same or a different company.

The organic carrier is preferably selected from an organic polymer or co-polymer.

The polymer can e.g. be a cellulose based polymer, acrylate, poloxamer, vinyl homopolymer or copolymer, polyethylene glycol, aminosaccharide or polyethylene oxide.

Examples of a cellulose based polymer include, but are not limited to alkylcelluloses, e.g., methylcellulose; hydroxyalkylcelluloses, e.g., hydroxymethylcellulose, hydroxyethylcellulose (Natrosol™, Ashland, Covington, Ky.), hydroxypropylcellulose, hydroxybutylcellulose and weakly substituted hydroxypropylcellulose; hydroxyalkylalkylcelluloses, e.g., ethyl(hydroxyethyl)cellulose, hydroxyethylmethylcellulose and hydroxypropylmethylcellulose (e.g., Methocel, types A, E, K, F, Dow Wolff Cellulosics GmbH, Bomlitz, Germany and Pharmacoat, types 603, 606, 615, 645, Harke Services GmbH, Muelheim an der Ruhr, Germany). Particularly preferred is HPMC of low viscosity.

Examples of acrylate include polyacrylates including, but are not limited to, methacrylic acid copolymer, polymethacrylates (Eudragit® L-100-55 and Eudragit® E-100, Evonik Degussa Corporation, Parsipanny, N.J.), polyacrylic acid (Carbopol®, The Lubrizol Corporation, Wickliffe, Ohio).

Examples of vinyl homopolymers and copolymers include, but are not limited to, polymers of N-vinylpyrrolidone, in particular povidone, copovidone, polyvinyl alcohol, and polyvinylpyrrolidone (Kollidon™, PVP and PVP-VA, BASF SE, Ludwigshafen, Germany).

Examples of other types of synthetic polymers include, but are not limited to, polyethylene oxide (Polyox™, Dow Chemical Company, Midland, Mich.), polyethyleneglycols of various molecular weights, polyethylene-/polypropylene-/polyethylene-oxide block copolymers and natural gums and polysaccharides—Xanthan gum (Keltrol™, CP Kelco, Atlanta, Ga.), carrageenan, locust bean gum, acacia gum, chitosan, alginic acid, hyaluronic acid, pectin, etc. Suitable polyethyleneglycols are especially Polyethyleneglycol 8000 and Polyethyleneglycol 6000. A suitable polyethylene-/polypropylene-/polyethylene-oxide block copolymer is in particular Pluronic F68.

It is particularly preferred that the organic polymer or co-polymer is selected from the list consisting of a hydroxyalkylcellulose, hydroxyalkylalkylcellulose, preferably HPMC and a polyvinylcaprolactam—polyvinyl acetate—polyethylene glycol graft copolymer. The polyvinylcaprolactam—polyvinyl acetate—polyethylene glycol graft copolymer can for example be obtained from BASF under the trade name Soluplus®.

The solid solution or solid dispersion of the present invention can consist exclusively of vortioxetine hydrobromide and the at least one organic carrier.

However, in a further preferred embodiment the solid solution or solid dispersion of the present invention contains vortioxetine hydrobromide, the at least one organic carrier and at least one further ingredient. Examples of categories of suitable ingredients include, but are not limited to antioxidants, binders, bulking agents, disintegrants, fillers, glidants, lubricants and surfactants. In general, the further ingredient is contained in an amount of about 0.01 to about 80%, preferably of about 5 to about 50% by weight relative to the weight of the solid solution or solid dispersion.

Examples of antioxidants include water soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butylated hydroxyanisole, butylated hydroxytoluene, potassium propyl gallate, octyl gallate, dodecyl gallate, phenyl-α-napthyl-amine, and tocopherols such as α-tocopherol.

Examples of binders include, but are not limited to, starches, celluloses and derivatives thereof, sucrose, dextrose, corn syrup, polysaccharides, and gelatin. Examples of celluloses and derivatives thereof include for example, microcrystalline cellulose, e.g., AVICEL PH from FMC (Philadelphia, Pa.). Particularly preferred is microcrystalline cellulose, e.g., AVICEL PH 200 from FMC (Philadelphia, Pa.).

Examples of bulking agents include, without limitation, PEGs, mannitol, trehalose, lactose, sucrose, sucrose, glycine, cyclodextrins, dextran and derivatives and mixtures thereof. Especially preferred is mannitol, e.g. PEARLITOL® 50C from Roquette Pharma (Lestrem, France).

Examples of disintegrants include, but are not limited to starches, e.g. sodium carboxymethyl starch or sodium starch glycolate; clays; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. Especially preferred is sodium starch glycolate, e.g. PRIMOJEL® from DFE-Pharma (Goch, Germany).

Examples of pharmaceutically fillers include, but are not limited to confectioner's sugar, compressible sugar, dextrates, dextrin, dextrose, lactose, mannitol, microcrystalline cellulose, powdered cellulose, sorbitol, sucrose and talc.

Examples of glidants and lubricants include, but are not limited to, colloidal silica, magnesium trisilicate, starches, talc, tribasic calcium phosphate, magnesium stearate, aluminum stearate, calcium stearate, magnesium carbonate, magnesium oxide and polyethylene glycol. Especially preferred is magnesium stearate.

Surfactants include, but are not limited to, fatty acid and alkyl sulfonates; benzethonium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1-palmitoyl-2-Sn-glycero-3-phosphocholine, lecithin and other phospholipids.

It is preferred that the solid dispersion or solid solution contains microcrystalline cellulose.

The organic carrier and “optional further ingredients” differ in that the organic carrier forms a continuous phase in which the active ingredient and the optional further ingredients are dispersed or dissolved.

The pharmaceutical composition of the present invention can consist exclusively of the solid solution or solid dispersion and in a preferred embodiment the pharmaceutical composition comprises only the solid solution or solid dispersion as defined above.

However, in a further preferred embodiment the pharmaceutical composition of the present invention contains the solid solution or solid dispersion and at least one pharmaceutically acceptable excipient. Examples of categories of suitable excipients include, but are not limited to antioxidants, binders, buffering agents, bulking agents, disintegrants, diluents, fillers, glidants, lubricants, preservatives, surfactants and cosurfactants. One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art. The following references disclose techniques and excipients used to formulate oral dosage forms (see The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2000).

Typical excipients include antioxidants. Antioxidants may be used to protect ingredients of the composition from oxidizing agents that are included within or come in contact with the composition. Examples of antioxidants include water soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butylated hydroxyanisole, butylated hydroxytoluene, potassium propyl gallate, octyl gallate, dodecyl gallate, phenyl-α-napthyl-amine, and tocopherols such as α-tocopherol.

Examples of pharmaceutically acceptable binders include, but are not limited to, starches, celluloses and derivatives thereof, copolymer of 1-vinyl-2-pyrrolidone and vinyl acetate, sucrose, dextrose, corn syrup, polysaccharides, and gelatin. Examples of celluloses and derivatives thereof include for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.), HP-Cellulose 100 (Klucel LF). Copolymer of 1-vinyl-2-pyrrolidone and vinyl acetate can be purchased as Kollidon VA64 from BASF. Especially preferred are hydroxypropylcellulose, e.g. KLUCEL EF from Ashland Inc. (Covington, USA) and microcrystalline cellulose, e.g., AVICEL PH 200 from FMC (Philadelphia, Pa.).

Buffering agents may be used to maintain an established pH of the composition. Examples of buffering agents included sodium citrate, calcium acetate, potassium metaphosphate, potassium phosphate monobasic, and tartaric acid.

Bulking agents are ingredients which may provide bulk to a pharmaceutical composition. Examples of bulking agents include, without limitation, PEGs, mannitol, trehalose, lactose, sucrose, polyvinyl pyrrolidone, sucrose, glycine, cyclodextrins, dextran and derivatives and mixtures thereof. Especially preferred is mannitol, e.g. PEARLITOL® 50C from Roquette Pharma (Lestrem, France).

Examples of pharmaceutically acceptable disintegrants include, but are not limited to, starches, e.g. sodium carboxymethyl starch or sodium starch glycolate; clays; celluloses, e.g. low substitute hydroxyl propyl cellulose; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. Especially preferred is sodium starch glycolate, e.g. PRIMOJEL® from DFE-Pharma (Goch, Germany).

Examples of pharmaceutically acceptable diluents and pharmaceutically acceptable fillers include, but are not limited to, confectioner's sugar, compressible sugar, dextrates, dextrin, dextrose, lactose, mannitol, microcrystalline cellulose, powdered cellulose, sorbitol, sucrose and talc.

Examples of pharmaceutically acceptable glidants and pharmaceutically acceptable lubricants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. Typically, a lubricant may be present in an amount from about 0.1% to about 5% by weight of the composition; whereas, the glidant, e.g., may be present in an amount from about 0.1% to about 10% by weight. Especially preferred is magnesium stearate.

Preservatives may also be used to protect the composition from degradation and/or microbial contamination. Examples of preservatives include liquipar oil, phenoxyethanol, methyl paraben, propyl paraben, butyl paraben, isopropyl paraben, isobutyl paraben, diazolidinyl urea, imidazolidinyl urea, diazolindyl urea, benzalkonium chloride, benzethonium chloride, phenol, and mixtures thereof (e.g., liquipar oil).

Surfactants are agents used to stabilize multi-phasic compositions, e.g., used as wetting agents, antifoam agents, emulsifiers, dispersing agents, and penetrants. Surfactants include, but are not limited to, fatty acid and alkyl sulfonates; benzethonium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1-palmitoyl-2-Sn-glycero-3-phosphocholine, lecithin and other phospholipids. Such surfactants, e.g., minimize aggregation of lyophilized particles during reconstitution of the product. Surfactants, if present, are typically used in an amount of from about 0.01% to about 5% w/w.

A cosurfactant is a surface-active agent that acts in addition to the surfactant by further lowering the interfacial energy but that cannot form micellar aggregates by itself. Cosurfactants can be, for example, hydrophilic or lipophilic. Examples of a cosurfactant include, but are not limited to, cetyl alcohol and stearyl alcohol.

In one embodiment of the invention, the amorphous vortioxetine hydrobromide is present in an amount of 1-80% (w/w) based on the total weight of the solid solution or solid dispersion. In a preferred embodiment, the amorphous vortioxetine hydrobromide is present in an amount of 10-70% (w/w) based on the total weight of the solid solution or solid dispersion. In another embodiment, the amorphous vortioxetine hydrobromide is present in an amount of 15-60% (w/w), preferably 20-50% (w/w), and most preferably 20-40% (w/w) based on the total weight of the solid solution or solid dispersion.

In one embodiment of the invention, the solid solution or solid dispersion is present in an amount of 1-100% (w/w) based on the total weight of the pharmaceutical composition. In a preferred embodiment, the solid solution or solid dispersion is present in an amount of 10-90 (w/w) based on the total weight of the pharmaceutical composition. In another embodiment, the solid solution or solid dispersion is present in an amount of 20-80% (w/w), preferably 40-80% (w/w), and most preferably 60-80% (w/w) based on the total weight of the pharmaceutical composition.

In one embodiment of the invention, the amorphous vortioxetine hydrobromide is present in an amount of 1-60% (w/w) based on the total weight of the pharmaceutical composition. In a preferred embodiment, the amorphous vortioxetine hydrobromide is present in an amount of 2-50% (w/w) based on the total weight of the pharmaceutical composition. In another embodiment, the amorphous vortioxetine hydrobromide is present in an amount of 5-40% (w/w), preferably 8-30% (w/w), and most preferably 10-20% (w/w) based on the total weight of the pharmaceutical composition.

Various techniques exist for preparing solid solutions or solid dispersions including solvent-evaporation, melt-extrusion and spray-drying. These methods are well known to a skilled person as disclosed in C. Leuner, J. Dressman/European Journal of Pharmaceutics and Biopharmaceutics 50 (2000) 47-60. Any of the well known methods for preparing solid solutions or solid dispersions can be used for preparing the solid solutions or solid dispersions of the present invention.

The vortioxetine hydrobromide that is used in the process for preparing the solid solution or solid dispersion can be crystalline or amorphous.

Any physical form of vortioxetine, such as e.g. crystalline hydrobromide salts (such as the alpha form or the beta form as disclosed in WO 2007/144005) or the crystalline hydrobromide salt as disclosed in WO 2014/044721, can be used to prepare a solid solution or solid dispersion of amorphous vortioxetine hydrobromide. Particularly preferred is the crystalline form disclosed in WO 2014/044721, i.e. a crystalline form of vortioxetine hydrobromide having an XRPD pattern with characteristic peaks (expressed in 2θ±0, 2° 2θ (CuKα radiation)) at 5.5°, 14.8°, 16.7° and 20.0°. A method of producing this crystalline form is disclosed in WO 2014/044721. Also preferred is the alpha form or the beta form as disclosed in WO 2007/144005.

If crystalline vortioxetine hydrobromide is used in the process of the invention it will be transformed to amorphous vortioxetine hydrobromide which is present in the solid solution or solid dispersion. Of course, in a solid solution, the vortioxetine hydrobromide is molecularly dispersed (dissolved) in the at least one organic carrier and for the purpose of this invention this is considered as an amorphous form of vortioxetine hydrobromide.

The solvent-evaporation method is widely used in the process of preparing solid solutions or solid dispersions. Generally, the active ingredient is dissolved or dispersed in a suitable solvent. In the next step, organic carriers, such as polymers and optional further ingredients are added. It is also possible to first dissolve or disperse the at least one organic carrier and optional further ingredients and then, in the next step to add the active ingredient.

Therefore, in one embodiment the solvent-evaporation process comprises the steps of dissolving or dispersing vortioxetine hydrobromide in a protic solvent, an aprotic solvent, or a mixture of a protic solvent and an aprotic solvent, adding the at least one organic carrier and optional further ingredients, mixing, e.g. in a fluidized bed, and removing the solvent.

In another embodiment, the solvent-evaporation process comprises the steps of dissolving or dispersing the at least one organic carrier and optional further ingredients in a protic solvent, an aprotic solvent, or a mixture of a protic solvent and an aprotic solvent, adding vortioxetine hydrobromide, mixing, e.g. in a fluidized bed, and removing the solvent.

Removing the solvent can be performed in different ways. For example, the mixture can be stirred at 900 rpm for at least 1 hour to evaporate the solvent. Depending on the solvent used, this can be carried out at room or elevated temperatures. There is also the possibility to remove the solvent under reduced pressure. Other solvent removing techniques are well known to those skilled in the art. Generally, the resultant solid solution or solid dispersion is then stored, e.g. for at least 24 hours, in desiccators in order to remove remaining traces of solvent.

Examples for the solvent are alcohols, aliphatic hydrocarbons or esters. Particularly preferred are methanol, ethanol, dichloromethane, isopropanol and acetone.

The melt-extrusion process comprises the steps of preparing a homogeneous melt of vortioxetine hydrobromide and the at least one organic carrier, and cooling the melt until it solidifies. “Melting” means a transition into a liquid or rubbery state in which it is possible for one component to get embedded homogeneously in the other. Typically, one component will melt and the other components will dissolve in the melt thus forming a solution. Melting usually involves heating above the softening point of the at least one organic carrier. The preparation of the melt can take place in a variety of ways. The mixing of the components can take place before, during or after the formation of the melt. For example, the components can be mixed first and then melted or be simultaneously mixed and melted. Usually, the melt is homogenized in order to disperse the active ingredients efficiently. Also, it may be convenient first to melt the at least one organic carrier and then to mix in and homogenize the vortioxetine hydrobromide.

Usually, the melt temperature is in the range of about 70 to about 250° C., preferably from about 100 to about 240° C., most preferred from about 110 to about 225° C.

The vortioxetine hydrobromide can be employed as such or as a solution or dispersion in a suitable solvent such as alcohols, aliphatic hydrocarbons or esters. The solvent is removed, e.g. evaporated, upon preparation of the melt.

Various additives may be included in the melt, for example flow regulators such as colloidal silica; lubricants, fillers, disintegrants, plasticizers, stabilizers such as antioxidants, light stabilizers, radical scavengers, stabilizers against microbial attack.

Moreover, it may be advantageous to mix a sugar alcohol and/or a cellulose into the polymer melt, for example Isomalt, mannitol, sorbitol or xylitol or microcrystalline cellulose. Then, the extruded mixture is subjected to spheronizing, pelletizing, milling or direct shaping.

Besides the solvent-evaporation technique and melt processes spray drying is also a common manufacturing method for solid solutions or solid dispersions. Suitable spray-drying techniques are described, for example, by K. Masters in “Spray Drying Handbook”, John Wiley & Sons, New York, 1984 and Remington's Pharmaceutical Sciences, edition 20, edited by A. R. Gennaro, Mack Publishing Co., 2000. Generally, the active ingredient is dissolved or dispersed in a suitable solvent. In the next step, organic carriers, such as polymers and optional further ingredients are added. It is also possible to first dissolve or disperse the at least one organic carrier and optional further ingredients and then, in the next step to add the active ingredient. Then, heat from a hot gas such as heated air or nitrogen is used to evaporate the solvent from droplets formed by atomizing a continuous liquid feed. Other spray-drying techniques are well known to those skilled in the art.

Therefore, in one embodiment, the spray-drying process comprises the steps of dissolving or dispersing vortioxetine hydrobromide in a protic solvent, an aprotic solvent, or a mixture of a protic solvent and an aprotic solvent, adding the at least one organic carrier and optional further ingredients, mixing, and spray-drying the solution.

In another embodiment, the spray-drying process comprises the steps of dissolving or dispersing the at least one organic carrier and optional further ingredients in a protic solvent, an aprotic solvent, or a mixture of a protic solvent and an aprotic solvent, adding vortioxetine hydrobromide, mixing, and spray-drying the solution.

Usually, the inlet air temperature during the spray drying process is in the range of 60-160° C. and the outlet air temperature is 40-90° C. The spraying rate of the liquid and the inlet air volume have to be adapted to obtain a certain required temperature profile. Thus, the inlet air temperature, spraying rate and inlet air volume strongly influence the outlet air temperature.

Typical solvents are for example alcohols, aliphatic hydrocarbons or esters. Particularly preferred are ethanol, isopropanol, methanol, acetone, 2-butanone, ethyl acetate, butyl acetate, tetrahydrofurane and methylene chloride.

In another embodiment the solid solution or solid dispersion is applied to a substrate. Suitable substrates are any substrates onto which a solid solution or solid dispersion can be sprayed, e.g. pellets, but also granules or even powders such as MCC powder or lactose powder (here the normal MCC or lactose qualities used as pharmaceutical excipients can be used). Examples for substrates are inert cores like sugar spheres, or MCC pellets, or inert powders such as lactose monohydrate, microcrystalline cellulose, Ca-phosphate and mannitol.

In a preferred embodiment the solid solution or solid dispersion consists exclusively of vortioxetine hydrobromide, the at least one organic carrier and the optional further ingredients.

In another preferred embodiment of the invention the vortioxetine hydrobromide is in association with an adsorbent. As used herein, the term “in association with” is intended to mean that the vortioxetine hydrobromide forms an adsorbate on the surface of the adsorbent. Examples of adsorbents include, but are not limited to SiO₂, Al₂O₃, TiO₂, MgO, synthetic and amorphous silicas, such as Aerosil®, for example Aerosil® 200, Aerosil® 380 (Evonik Industries), Syloid®, for example Syloid® AL1, Syloid® 72 FP and Syloid® 244 FP (W.R. Grace & Co.-Conn), and synthetic and amorphous silicates such as Neusilin® and in particular Neusilin® UFL2 (Fuji Chemical industry Co., Ltd.). Then, the vortioxetine hydrobromide in association with an adsorbent is processed into a solid solution or solid dispersion according to one of the processes described above.

The pharmaceutical composition described above can be used in the treatment of a disease selected from affective disorders, depression, major depressive disorder, postnatal depression, depression associated with bipolar disorder, Alzheimer's disease, psychosis, cancer, age or Parkinson's disease, anxiety, general anxiety disorder, social anxiety disorder, obsessive compulsive disorder, panic disorder, panic attacks, phobia, social phobia, agoraphobia, stress urinary incontinence, emesis, irritable bowel syndrome, eating disorders, chronic pain, partial responders, treatment resistant depression, Alzheimer's disease, cognitive impairment, attention deficit hyperactivity disorder, melancholia, posttraumatic stress disorder, hot flushes, sleep apnea, alcohol, nicotine or carbohydrate craving, substance abuse and alcohol or drug abuse.

For the above-mentioned indications, the appropriate dosage will vary depending on, for example, the host, the mode of administration, the nature and severity of the condition, disease or disorder or the effect desired. Amorphous vortioxetine hydrobromide may be conveniently administered in a unit dose form comprising about 1 to 50 mg of amorphous vortioxetine hydrobromide. The total daily dose is expected to be in the range of about 1 to 20 mg of amorphous vortioxetine hydrobromide.

The present invention also comprises a method for stabilizing a pharmaceutical composition comprising amorphous vortioxetine hydrobromide, characterized in that the amorphous vortioxetine hydrobromide is formulated in a solid solution or solid dispersion in at least one organic carrier.

Herein, “stabilizing” means that amorphous vortioxetine hydrobromide remains in the amorphous state in the solid solution or solid dispersion for at least 1 week, preferably at least 2 weeks, and most preferably at least 6 weeks at room temperature. The amorphous state of vortioxetine hydrobromide is preferably determined by XRPD using a method as disclosed later herein.

Stability also means that amorphous vortioxetine hydrobromide is chemically stable in the solid solution or solid dispersion during storage and meets the requirements of the European Pharmacopoeia, preferably it is much more stable than required by the Pharmacopoeia.

The pharmaceutical compositions of the present invention are preferably in solid oral dosage form. Solid oral dosage forms include, but are not limited to tablets, hard or soft capsules, caplets, lozenges, pills, mini-tablets, pellets, beads, granules (e.g. packaged in sachets), or powders. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations.

Tablets may be either film coated or enteric coated according to methods known in the art. Tablets can be optionally coated with a functional or non-functional coating as known in the art. Examples of coating techniques include, but are not limited to, sugar coating, film coating, microencapsulation and compression coating. Types of coatings include, but are not limited to, enteric coatings, sustained release coatings, controlled-release coatings. Anhydrous pharmaceutical compositions and dosage forms can also be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

As used herein, a unit dosage form is a single dosage form which has the capacity of being administered to a subject to be effective, and which can be readily handled and packaged, remaining as a physically and chemically stable unit dose comprising the active ingredient.

Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

A hard gelatin capsule, also known as a dry-filled capsule, is composed of two sections, one slipping over the other, thus completely surrounding (encapsulating) the drug formulation. A soft elastic capsule has a soft, globular, e.g., gelatin shell.

The XRPD's can be obtained according to the following XRPD method:

The X-ray powder diffractograms (XRPD) were obtained with an X'Pert PRO diffractometer (PANalytical, Almelo, The Netherlands) equipped with a theta/theta coupled goniometer in 25 transmission geometry, programmable XYZ stage with well plate holder, Cu-Kα1,2 radiation source (wavelength 0.15419 nm) and a solid state PIX'cel detector. The diffractograms were recorded at a tube voltage of 40 kV, tube current of 40 mA. A typical precision of the 2-theta values is in the range of about ±0.2° 2-theta. Thus a diffraction peak that appears at 5.0° 2-theta can appear between 4.8 and 5.2° 2-theta on most X-ray 30 diffractometers under standard conditions.

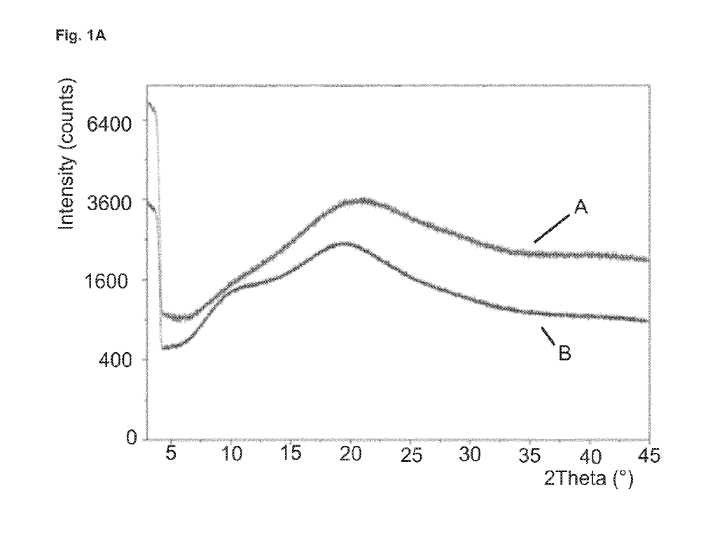

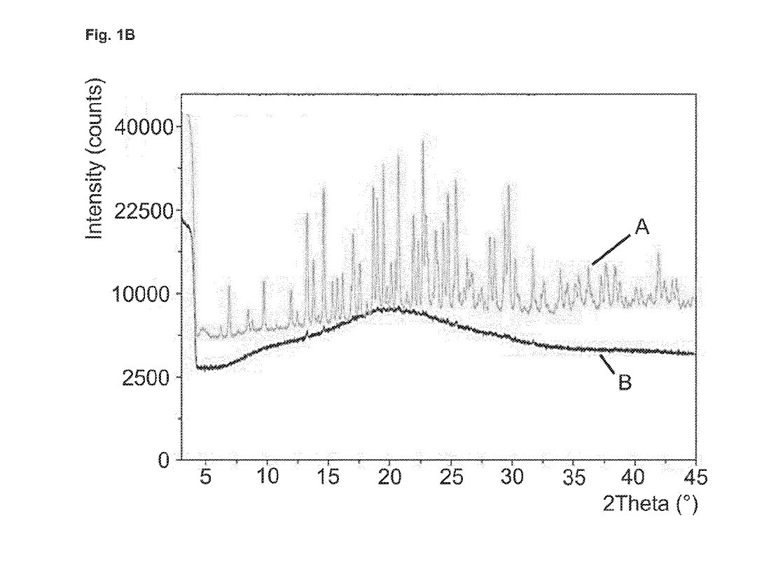

FIG. 1 relates to solid dispersions prepared by hot melt extrusion according to example 1.

FIG. 1A shows XRPD patterns of vortioxetine hydrobromide in Soluplus as organic carrier (A) and of Soluplus without vortioxetine hydrobromide (B).

FIG. 1B shows XRPD patterns of vortioxetine hydrobromide crystalline form β (A) and the product obtained in example 1 after milling the melt extruded product (B).

FIG. 1C shows XRPD patterns of the final tablets prepared in example 1 (A) and of correspondingly prepared tablets without vortioxetine hydrobromide (B).

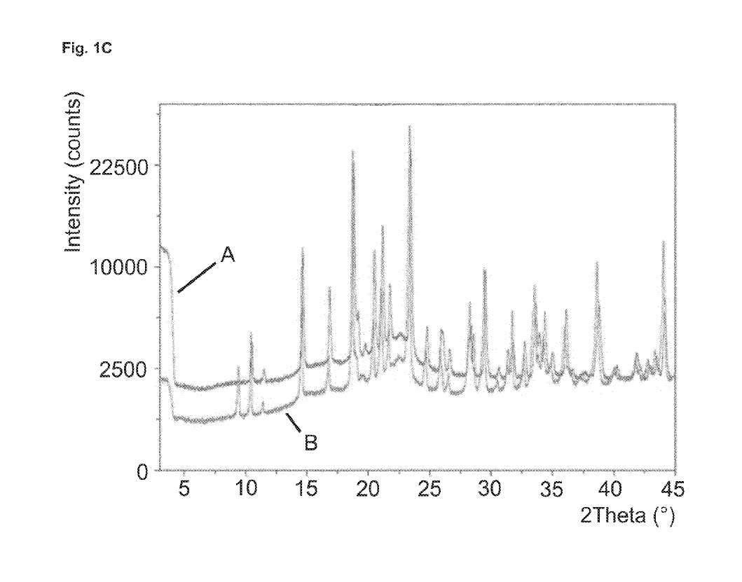

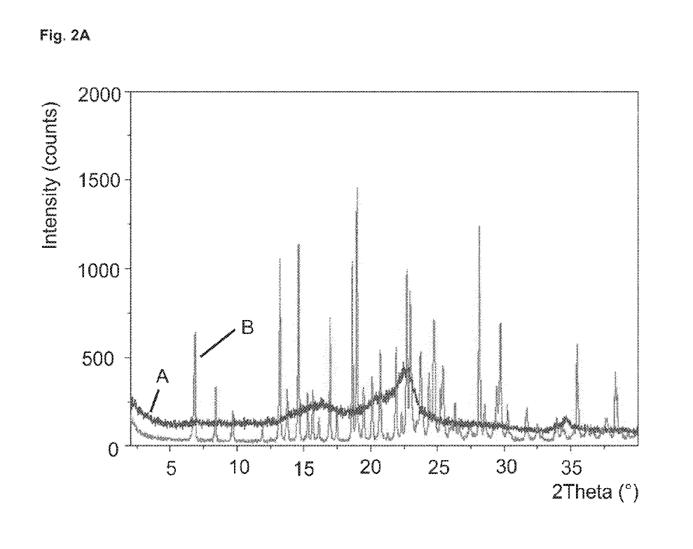

FIG. 2 relates to solid dispersions with HPMC prepared by solvent evaporation according to example 2

FIG. 2A shows XRPD patterns of vortioxetine hydrobromide crystalline form β (B) and of the end product of example 2 (A).

FIG. 2B shows XRPD patterns of vortioxetine hydrobromide crystalline form β (C) and of the solid dispersions with HPMC immediately after the preparation (A) and after 1 week at room temperature (B).

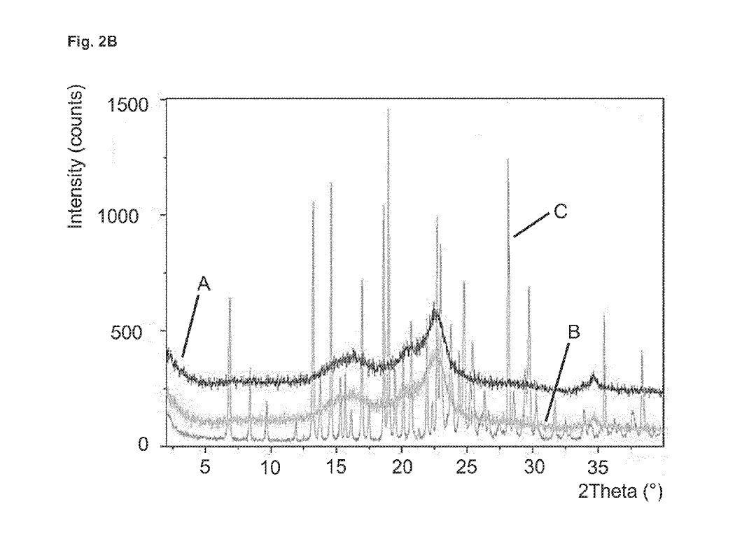

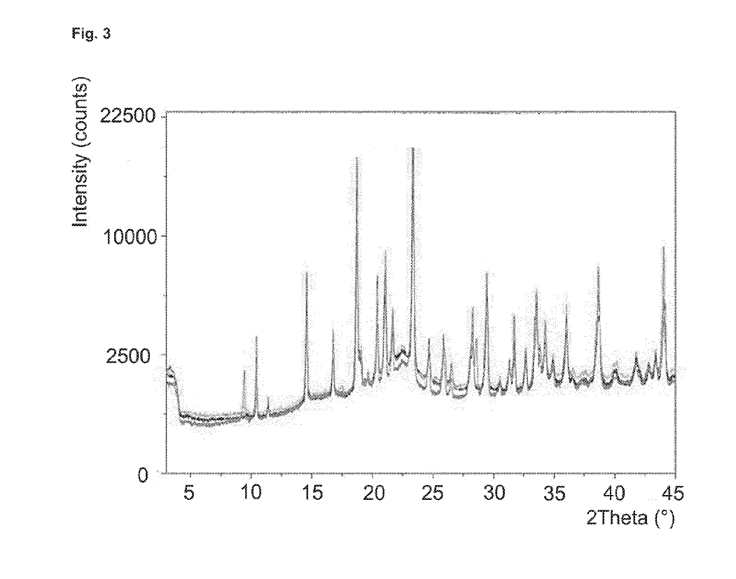

FIG. 3 shows XRPD patterns for compressed tablets with adsorbate Neusilin US2/adsorbate Syloid 244FP according to reference examples 6+7

The present invention will be explained in more detail with the following examples, which are not to be interpreted as limiting.

### EXAMPLES

### Example 1: Solid Solution/Dispersion Prepared by Hot Melt Extrusion

Any physical form of vortioxetine (e.g., produced as described in WO 2003/029232, WO 2007/144005 or WO 2014/044721) can be used to prepare a solid solution or solid dispersion of amorphous vortioxetine hydrobromide.

5.0 g of the vortioxetine hydrobromide salt and 15.0 g of Soluplus (polyvinyl caprolactam—polyvinyl acetate—polyethylenglycole graft copolymer) were mixed for 10 min in a suitable bin blender. The mixture was passed through a DSM Xplore 5×15 Micro Compounder at 225° C. and 50 rpm. 1.24 g of the resulting granules were passed through a mill with screen 1.0 mm and mixed together with 12.2 g Mannitol, 1.0 g hydroxypropyl cellulose (KLUCEL EF), 5.08 g microcrystalline cellulose and 0.98 g sodium starch glycolate (type A). Following lubrication of the blend by mixing with 0.26 g magnesium stearate the powder blend was transferred to a tablet press. Tablets having a target core weight of 125 mg and a diameter of 6.5 mm were prepared to obtain tablets with a target content of the hydrobromide salt corresponding to 5 mg of the free base.

In FIGS. 1A, 1B and 1C it has been shown that the solid solution/dispersion obtained in example 1 contains amorphous vortioxetine hydrobromide.

FIG. 1A shows XRPD's of Soluplus alone (B) and of the solid solution/dispersion (A) obtained after the melt extrusion. It can be seen that no crystalline compounds are present.

FIG. 1B shows XRPD's of vortioxetine hydrobromide crystalline form β (A) and of the solid solution/dispersion after the milling step (B). It can be seen that the vortioxetine hydrobromide in the solid solution/dispersion stays in the amorphous form even after the milling step.

FIG. 1C shows the XRPD's of a blank tablet (no vortioxetine hydrobromide) (B) and of the corresponding tablet containing vortioxetine hydrobromide (A). Both tablets were prepared by the method of example 1. It can be seen, that the XRPD's of both tablets are identical, which means that the vortioxetine hydrobromide in the finished tablets is amorphous.

### Example 2: Solid Solution/Dispersion with HPMC Prepared by Solvent Evaporation

1 g of the vortioxetine hydrobromide salt and 2 g of hydroxypropylmethylcellulose (Pharmacoat 603) were solved under stirring in an appropriate amount of methanol; after clear solution occurred 3 g of microcrystalline cellulose were added and stirred over a period of 30 min until homogenous suspension was reached. The solvent was removed by evaporation under rotation followed by vacuum drying at 40° C./25 mbar 24 h.

In FIGS. 2A and 2B it has been shown that the solid solution/dispersion obtained in example 2 contains amorphous vortioxetine hydrobromide.

FIG. 2A shows XRPD's of vortioxetine hydrobromide crystalline form β (B) and of the solid solution/dispersion (A). It can be seen that the vortioxetine hydrobromide in the solid solution/dispersion is amorphous.

FIG. 2B shows XRPD's of vortioxetine hydrobromide crystalline form β (C), of the solid solution/dispersion immediately after preparation (A) and after 1 week at room temperature (B). It can be seen, that the vortioxetine hydrobromide in the solid solution/dispersion immediately after preparation and also after storage for 1 week at room temperature is amorphous.

### Example 3: Solid Solution/Dispersion with HPMC Prepared with Fluid Bed Granulation

100 g of the vortioxetine hydrobromide salt and 200 g of hydroxypropylmethylcellulose (Pharmacoat 603) is dissolved under stirring in 1667 ml methanol; the solution is sprayed on 300 g of microcrystalline cellulose during continuous fluidization in a fluid bed granulation Typ Glatt GPCG 1. The final granules are further treated over 24 h at 40° C./25 mbar in a vacuum chamber.

### Example 4: Solid Dispersion with an Adsorbate

The adsorbate of vortioxetine hydrobromide on magnesium aluminometasilicate grade US2 (Neusilin US2) was prepared as described in Example 8 (amorphous vortioxetine HBr on Neusilin US2 and HPMC).

11.62 g of the solid dispersion, 25.03 g Mannitol, 2.0 g HPC (KLUCEL EF), 18.8 g microcrystalline cellulose and 2.02 g Sodium Starch glycolate (Type A) were mixed for 20 min in a suitable bin blender. Following lubrication of the blend by mixing with 0.6 g magnesium stearate the powder blend was transferred to a tablet press. Tablets having a target core weight of 150 mg and a diameter of 8 mm were prepared to obtain tablets with a target content of the hydrobromide salt corresponding to 5 mg of the free base.

### Example 5: Solid Dispersion with an Adsorbate

The adsorbate of vortioxetine hydrobromide on magnesium aluminometasilicate grade US2 (Neusilin US2) was prepared as described in Example 8 (amorphous vortioxetine HBr on Neusilin US2 and HPMC).

12.06 g of the solid dispersion, 25.90 g Mannitol and 2.08 g hydroxypropylcellulose (KLUCEL EF) were mixed for 2 min in a suitable mixing device. 18.1 g water were added under continuous stirring over 2 min, followed by 2 min kneading. The wet mass were sieved through a sieve of 2 mm, followed by tray drying at 42° C. over 1 h. 30.8 g of the resulting granules were mixed together with 15.08 g microcrystalline cellulose, 1.62 g Sodium starch glycolate for 10 min. Following lubrication of the blend by mixing with 0.48 g magnesium stearate the powder blend was transferred to a tablet press. Tablets having a target core weight of 150 mg and a diameter of 7 mm were prepared to obtain tablets with a target content of the hydrobromide salt corresponding to 5 mg of the free base.

### Example 6: Adsorbate (Neusilin US2) Tablets (Reference)

The adsorbate of vortioxetine hydrobromide was prepared as described in Example 8.

23.88 g of the vortioxetine hydrobromide adsorbate (vortioxetine hydrobromide on magnesium aluminometasilicate grade US2 (Neusilin US2)), 70.50 g Mannitol, 5.67 g hydroxypropylcellulose (KLUCEL EF), were mixed for 2 min in a suitable mixing device. 50.0 g water were added under continuous stirring over 5 min, followed by 2 min kneading. The wet mass were sieved through a sieve of 2 mm, followed by tray drying at 42° C. over 90 min. 81.70 g of the resulting granules were mixed together with 27.75 g microcrystalline cellulose, 4.64 g Sodium starch glycolate for 10 min. Following lubrication of the blend by mixing with 1.15 g magnesium stearate the powder blend was transferred to a tablet press. Tablets having a target core weight of 125 mg and a diameter of 6.5 mm were prepared to obtain tablets with a target content of the hydrobromide salt corresponding to 5 mg of the free base.

### Example 7: Adsorbate (Syloid 244FP) (Reference)

The adsorbate of vortioxetine hydrobromide was prepared as described in Example 8.

23.9 g of the vortioxetine hydrobromide adsorbate (vortioxetine hydrobromide on silica (Syloid 244FP)), 70.47 g Mannitol, 5.70 g hydroxypropylcellulose (KLUCEL EF), were mixed for 2 min in a suitable mixing device. 45.0 g water were added under continuous stirring over 5 min, followed by 2 min kneading. The wet mass were sieved through a sieve of 2 mm, followed by tray drying at 42° C. over 80 min. 82.90 g of the resulting granules were mixed together with 28.15 g microcrystalline cellulose, 4.68 g Sodium starch glycolate for 10 min. Following lubrication of the blend by mixing with 1.18 g magnesium stearate the powder blend was transferred to a tablet press. Tablets having a target core weight of 125 mg and a diameter of 6.5 mm were prepared to obtain tablets with a target content of the hydrobromide salt corresponding to 5 mg of the free base.

FIG. 3 shows XRPD's of the tablets obtained in examples 6 and 7 and XRPD's of corresponding blank tablets without vortioxetine hydrobromide. All XRPD's are essentially identical showing that the adsorbates contain amorphous vortioxetine hydrobromide.

### Example 8

Preparation methods of vortioxetine HBr adsorbates are presented below. Alternatively, the methods according to the non-prepublished copending patent application PCT/EP2014/058546 may be used (incorporated herein with reference).

### Preparation of Amorphous Vortioxetine HBr on Neusilin US2

Vortioxetine HBr (1.0 g) was dissolved in dichloromethane (100 mL) at room temperature. In the obtained clear solution Neusilin US2 was added. The mixture was further stirred for 1 h and then the solvent was completely evaporated on a rotary evaporator under reduced pressure at room temperature. The dry product was analyzed by DSC and PXRD and found to be amorphous as shown in Table 1 below.

TABLE 1

| Neusilin US2 | Loading | |
|---|---|---|
| [g] | [%] | Form |
| 2.30 | 30 | amorphous |
| 2.05 | 33 | amorphous |
| 1.65 | 38 | amorphous |

^{a} Evaporator under reduced pressure at a bath temperature 40° C.

### Preparation of Amorphous Vortioxetine HBr on Neusilin US2

Vortioxetine HBr (1.0 g) was dissolved in dichloromethane (30 mL) at reflux. In the obtained clear solution Neusilin US2 was added, heating was then turned off. Obtained mixture was stirred for 19 h at room temperature and then the slurry was filtered. The cake was dried overnight in vacuum at 30° C. The dry product was analyzed by DSC and PXRD and found to be amorphous as shown in Table 2 below.

TABLE 2

| Neusilin US2 | Loading | |
|---|---|---|
| [g] | [%] | Form |
| 1.5 | 29 | amorphous |
| 1.0 | 30 | amorphous |

### Preparation of Amorphous Vortioxetine HBr on Neusilin US2 and HPMC

Vortioxetine HBr (7.6 g) was dissolved in dichloromethane (380 mL) at room temperature. In the obtained clear solution Neusilin US2 (18.7 g) and HPMC (7.7 g) were added. The mixture was further stirred for 1.5 h and then the solvent was completely evaporated on a rotary evaporator under reduced pressure at room temperature. The dry product was analyzed by DSC and PXRD and found to be amorphous.

### Preparation of Amorphous Vortioxetine HBr on Syloid 244 FP

Vortioxetine HBr was dissolved in dichloromethane at room temperature. In the obtained clear solution Syloid 244 FP was added. The mixture was further stirred for a certain time and then the solvent was completely evaporated on a rotary evaporator under reduced pressure. The dry product was analyzed by DSC and PXRD and found to be amorphous as shown in Table 3 below.

TABLE 3

| Vortioxetine | Syloid | | | | |
|---|---|---|---|---|---|
| HBr | 244 FP | CH₂Cl₂ | Stirring time | Loading | |
| [g] | [g] | [mL] | [h] | [%] | Form |
| 0.2 | 0.3 | 20 | 21 | 40 | amorphous |
| 1 | 2.3 | 50 | 1 | 30 | amorphous |

### Preparation of Amorphous Vortioxetine HBr on Syloid 244 FP

Vortioxetine HBr (0.2 g) was dissolved in dichloromethane (10 mL) at room temperature. In the obtained clear solution Syloid 244 FP was added. Obtained mixture was stirred at room temperature for a certain time and then the slurry was filtered. The cake was dried overnight in vacuum at 30° C. The dry product was analyzed by DSC and PXRD and found to be amorphous as shown in Table 4 below.

TABLE 4

| Neusilin US2 | Stirring time | Loading | |
|---|---|---|---|
| [g] | [h] | [%] | Form |
| 0.2 | 7 | 30 | amorphous |
| 1.3 | 26 | 38 | amorphous |

1. A pharmaceutical composition comprising a solid solution or solid dispersion of amorphous vortioxetine hydrobromide in at least one organic carrier, and at least one further ingredient being contained in an amount of about 0.01 to about 80% by weight relative to the weight of the solid solution or solid dispersion.

2. The pharmaceutical composition according to claim 1, wherein the organic carrier is selected from an organic polymer or co-polymer.

3. The pharmaceutical composition according to claim 1, wherein the organic carrier comprises at least one compound selected from the group consisting of a hydroxyalkylcellulose, hydroxyalkylalkycellulose, and a polyvinylcaprolactam—polyvinyl acetate—polyethylene glycol graft copolymer.

4. The pharmaceutical composition according to claim 1, wherein the solid solution or solid dispersion further comprises microcrystalline cellulose.

5. The pharmaceutical composition according to claim 1, wherein the solid solution or solid dispersion is applied to a substrate.

6. The pharmaceutical composition according to claim 1, wherein the amorphous vortioxetine hydrobromide is present in an amount of 20-50% (w/v), based on the total weight of the solid solution or solid dispersion.

7. A process for the preparation of the pharmaceutical composition according to claim 1, comprising a process step selected from
(i) subjecting vortioxetine hydrobromide, the at least one organic carrier and optional further ingredients to solvent-evaporation;
(ii) subjecting vortioxetine hydrobromide, the at least one organic carrier and optional further ingredients to melt extrusion; and
(iii) spray drying a solution or dispersion comprising vortioxetine hydrobromide, the at least one organic carrier and optional further ingredients;
in order to obtain a solid solution or solid dispersion.

8. The process according to claim 7, said process comprising
(i) dissolving or dispersing vortioxetine hydrobromide and the at least one organic carrier and optional further ingredients in a protic solvent, an aprotic solvent, or a mixture of a protic solvent and an aprotic solvent;
(ii) mixing in a fluidized bed; and
(iii) removing the solvent.

9. The process according to claim 7, said process comprising
(i) mixing vortioxetine hydrobromide with the at least one organic carrier and optional further ingredients;
(ii) melt-extruding the mixture; and
(iii) subjecting the extrudate to spheronizing, pelletizing, milling or direct shaping.

10. The process according to claim 7, said process comprising
(i) dissolving or dispersing vortioxetine hydrobromide and the at least one organic carrier and optional further ingredients in a protic solvent, an aprotic solvent, or a mixture of a protic solvent and an aprotic solvent;
(ii) mixing; and
(iii) spray drying the mixture.

11. The process according to claim 7, wherein the solid solution or solid dispersion is applied to a substrate.

12. A pharmaceutical composition according to claim 7 for use in the treatment of a disease selected from affective disorders, depression, major depressive disorder, postnatal depression, depression associated with bipolar disorder, Alzheimer's disease, psychosis, cancer, age or Parkinson's disease, anxiety, general anxiety disorder, social anxiety disorder, obsessive compulsive disorder, panic disorder, panic attacks, phobia, social phobia, agoraphobia, stress urinary incontinence, emesis, irritable bowel syndrome, eating disorders, chronic pain, partial responders, treatment resistant depression, Alzheimer's disease, cognitive impairment, attention deficit hyperactivity disorder, melancholia, posttraumatic stress disorder, hot flushes, sleep apnea, alcohol, nicotine or carbohydrate craving, substance abuse and alcohol or drug abuse.

13. A method for stabilizing amorphous vortioxetine hydrobromide in a pharmaceutical composition, characterized in that the amorphous vortioxetine hydrobromide is formulated in a solid solution or solid dispersion in at least one organic carrier, and at least one further ingredient being contained in an amount of about 0.01 to about 80% by weight relative to the weight of the solid solution or solid dispersion.
